(19) United States
(12) Patent Application Publication
(10) Pub. No.: US 2023/0098559 A1
(43) Pub. Date: Mar. 30, 2023

(54) CURABLE COMPOSITION, CURED PRODUCT, ELECTRONIC DEVICE, DISPLAY DEVICE, OPTICAL MEMBER, POLYMER, PHOTOSENSITIVE COMPOSITION, PATTERN, AND COMPOUND

(71) Applicant: CENTRAL GLASS COMPANY, LIMITED, Ube-shi, Yamaguchi (JP)

(72) Inventors: Yuzuru KANEKO, Kawagoe-shi, Saitama (JP); Takashi AOKI, Kawagoe-shi, Saitama (JP); Yuki IMAIZUMI, Kawagoe-shi, Saitama (JP); Hiromasa IWATA, Kawagoe-shi, Saitama (JP)

(21) Appl. No.: 17/759,828

(22) PCT Filed: Feb. 3, 2021
(86) PCT No.: PCT/JP2021/003835

§ 371 Date: Jul. 29, 2022

(30) Foreign Application Priority Data
Feb. 7, 2020 (JP) 2020-019398
Sep. 8, 2020 (JP) 2020-150682

(51) Int. Cl. C08G 65/22; C08G 59/24; C08L 63/04; C08J 3/28

(52) U.S. Cl. CPC C08G 65/226; C08G 59/245; C08L 63/04; C08J 3/28; C08L 2201/10; C08L 2203/20

(57) ABSTRACT

A curable composition including a first compound having a group represented by general formula (x) and an epoxy group and having a molecular weight of 1000 or less. In the general formula (x), Rf₁ and Rf₂ each independently represent a fluorine-containing alkyl group.

—C(Rf₁)(Rf₂)—OH (x)

### TECHNICAL FIELD

The present invention relates to a curable composition, a cured product, an electronic device, a display device, an optical member, a polymer, a photosensitive composition, a pattern, and a compound.

### BACKGROUND ART

Unlike acrylic resins, epoxy resins are resins that have excellent moldability without curing shrinkage during polymerization or film formation and have excellent adhesion to a base material. Therefore, the epoxy resins are used in a wide range of applications.

Fluorine-based compounds have been continuously developed or used in a wide range of application fields, mainly in the field of advanced materials, due to characteristics of fluorine, such as water repellency, oil repellency, low water absorption, heat resistance, weather resistance, corrosion resistance, transparency, photosensitivity, low refractive index property, and low dielectric property. Specifically, in coating applications, active researches and developments have been made in the fields of anti-reflection films to which a low refractive index property and transparency of visible light are applied, optical devices to which transparency in a long wavelength band (an optical communication wavelength band) is applied, resist materials to which transparency in an ultraviolet region (particularly, a vacuum ultraviolet region) is applied, and the like.

For example, Patent Document 1 discloses a novel polymerizable monomer having high transparency in a wide wavelength region, that is, from a vacuum ultraviolet region to an optical communication wavelength band and having adhesion to a substrate and high film forming properties, a polymer compound using the same, an anti-reflection material coated with the polymer compound, an optical device material, and a resist material. As specific compounds, a series of novel fluorine-containing acrylate derivatives and monomers thereof, which have a high fluorine content and hydroxy groups, are described.

Patent Document 2 discloses a method for preparing a polyfluorinated polyether by allowing a fluorinated epoxide to react with a basic compound.

### RELATED DOCUMENT

### Patent Document

[Patent Document 1] Japanese Patent No. 4083399

[Patent Document 2] Japanese Patent No. 4570788

### SUMMARY OF THE INVENTION

### Technical Problem

The fluorine-containing acrylate derivatives described in Patent Document 1 and the polyfluorinated polyethers of Patent Document 2 do not have sufficient adhesion to a base material. Specifically, the adhesion to a substrate such as a silicon wafer, a glass substrate, a metal such as copper or silver is not sufficient. Then, there is room for improvement when used as a permanent film and when used as an adhesive and a wiring material.

The present inventors have conducted intensive studies in view of the above problems. Through the studies, the present inventors have newly found that the curable composition including a first compound described later has excellent adhesion to a base material. Then, based on this new finding, the present invention has been completed.

### Solution to Problem

According to the present invention, the following curable composition is provided.

A curable composition including: a first compound having a group represented by general formula (x) and an epoxy group, and having a molecular weight of 1000 or less.

−C(Rf₂)(Rf₂)—OH (x)

In the general formula (x), Rf₂ and Rf₂ each independently represent a fluorine-containing alkyl group.

Further, according to the present invention, a cured product of the above curable composition is provided.

Further, according to the present invention, an electronic device including the above cured product is provided.

Further, according to the present invention, a display device including the above cured product is provided.

Further, according to the present invention, an optical member including the above cured product is provided.

Further, according to the present invention, a polymer including a structural unit represented by general formula (2) is provided.

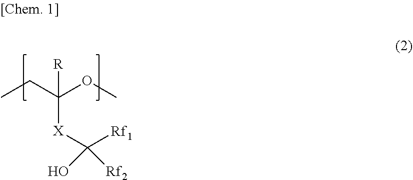

In the general formula (2),

R represents a hydrogen atom or a monovalent organic group,

X represents a divalent organic group,

Rf₁ represents a fluorine-containing alkyl group, and

Rf₂ represents a fluorine-containing alkyl group.

Further, according to the present invention, a compound represented by general formula (1) is provided.

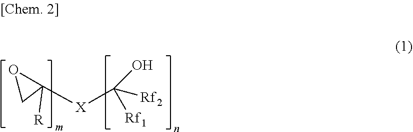

In the general formula (1),

R represents a hydrogen atom or a monovalent organic group,

X represents a (m+n)-valent organic group,

m is 1 to 4,

n is 1 to 4, and

Rf₁ and Rf₂ each independently represent a fluorine-containing alkyl group.

### Advantageous Effects of Invention

According to the present invention, a polymerizable composition having excellent adhesion to a base material is provided.

### DESCRIPTION OF EMBODIMENTS

Hereinafter, embodiments of the present invention will be described.

In the present specification, the notation “X to Y” in the description of the numerical range indicates X or more and Y or less unless otherwise specified. For example, “1% to 5% by mass” means “1% by mass or more and 5% by mass or less”.

In a case where substitution or unsubstitution is not noted in regard to the notation of a group (atomic group) in the present specification, the group includes not only a group not having a substituent but also a group having a substituent. For example, the concept of an “alkyl group” includes not only an alkyl group not having a substituent (unsubstituted alkyl group) but also an alkyl group having a substituent (substituted alkyl group).

Unless otherwise specified, the term “organic group” as used in the present specification means an atomic group obtained by removing one or more hydrogen atoms from an organic compound. For example, a “monovalent organic group” refers to an atomic group obtained by removing one hydrogen atom from a random organic compound.

The term “electronic device” in the present specification is used as a meaning including an element, a device, a final product, and the like, to which electronic engineering technology is applied, such as a semiconductor chip, a semiconductor element, a printed wiring board, an electric circuit display device, an information communication terminal, a light emitting diode, a physical battery, and a chemical battery.

<Curable Composition>

A curable composition of the present embodiment includes a first compound having a group represented by general formula (x) and an epoxy group and having a molecular weight of 1000 or less.

—C(Rf₂)(Rf₂)—OH (x)

In the general formula (x), Rf₂ and Rf₂ each independently represent a fluorine-containing alkyl group.

From the viewpoint of application to the curable composition, preferable embodiments of the first compound are as follows.

The first compound may have only one group represented by the general formula (x), or may have two or more groups. The number of groups represented by the general formula (x) in one molecule of the first compound is, for example, 1 to 4, preferably 1 to 2, and more preferably 1.

The first compound may have only one epoxy group or may have two or more epoxy groups. The number of epoxy groups in one molecule of the first compound is, for example, 1 to 4, preferably 1 to 2, and more preferably 1.

The molecular weight of the first compound is preferably 750 or less, more preferably 500 or less, still more preferably 400 or less, and particularly preferably 300 or less.

The first compound is usually neither a polymer nor an oligomer. That is, the first compound is not usually a compound obtained by polymerizing a monomer.

More specifically, the first compound preferably includes a compound represented by general formula (1).

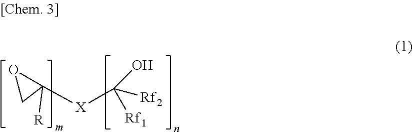

In the general formula (1),

R represents a hydrogen atom or a monovalent organic group,

X represents a (m+n)-valent organic group,

m is 1 to 4,

n is 1 to 4, and

Rf₁ and Rf₂ each independently represent a fluorine-containing alkyl group.

A cured product of the polymerizable composition of the present embodiment has excellent adhesion to a base material due to the epoxy group.

Further, the cured product of the polymerizable composition of the present embodiment has a low refractive index property due to the fluorine-containing alkyl group of the first compound. Therefore, the polymerizable composition of the present embodiment can be suitably used as a material for preparing a resin film constituting an electronic device or an optical member.

Further, the first compound included in the polymerizable composition of the present embodiment has excellent stability even though the first compound is a compound having a hydroxyl group and an epoxy group. It is considered that this is because the reaction between the hydroxyl group and the epoxy group is controlled by the steric hindrance of the fluorine-containing alkyl group of the first compound. Therefore, the polymerizable composition of the present embodiment has little change over time and is excellent in storage stability, ease of handling, and workability.

R in the general formula (1) represents a hydrogen atom or a monovalent organic group, and examples of the monovalent organic group include an alkyl group and an alkoxy group having 1 to 10 carbon atoms.

Examples of the alkyl group include a methyl group, an ethyl group, an n-propyl group, an isopropyl group, an n-butyl group, an isobutyl group, a sec-butyl group, a tert-butyl group, a pentyl group, a neopentyl group, a hexyl group, a heptyl group, an octyl group, a nonyl group, and a decyl group. Among these, a methyl group and an ethyl group are preferable.

Examples of the alkoxy group include a methoxy group, an ethoxy group, an n-propoxy group, an i-propoxy group, an n-butoxy group, an s-butoxy group, an isobutoxy group, and a t-butoxy group. Among these, a methoxy group and an ethoxy group are preferable.

m in the general formula (1) represents 1 to 4, preferably represents 1 or 2, and more preferably represents 1.

n in the general formula (1) represents 1 to 4, preferably represents 1 or 2, and more preferably represents 1.

X in the general formula (1) represents a (m+n)-valent organic group, preferably a monovalent to tetravalent organic group, and more preferably represents a divalent organic group. Examples of the divalent organic group include linear or branched alkylene groups having 1 to 6 carbon atoms. X preferably represents a linear alkylene group having 1 to 3 carbon atoms, and more preferably represents a methylene group (—CH₂—).

Rf₁ and Rf₂ in the general formula (1) each represent a fluorine-containing alkyl group and may be the same as or different from each other. The fluorine-containing alkyl group is preferably a linear or branched fluoroalkyl group having 1 to 10 carbon atoms, more preferably a linear or branched fluoroalkyl group having 1 to 6 carbon atoms, and even more preferably a linear or branched fluoroalkyl group having 1 to 3 carbon atoms. Among these, the fluorine-containing alkyl group is preferably a perfluoroalkyl group. As a result, a fluorine-containing epoxy compound represented by the general formula (1) can have a low refractive index property. Among these, Rf₁ and Rf₂ each preferably represent a perfluoromethyl group, a perfluoroethyl group, and a perfluorobutyl group, and particularly preferably represent a perfluoromethyl group (trifluoromethyl group).

The fluorine content of the first compound is preferably 25% by mass or more and 60% by mass or less, and more preferably 30% by mass or more and 55% by mass or less. When the fluorine content is within the above range, the cured product of the curable composition has excellent durability and can have a low refractive index property.

The cured product of the polymerizable composition of the present embodiment may have high adhesion to a base material. The cured product of the polymerizable composition of the present embodiment is usually formed from a polymer produced by a polymerization reaction involving the first compound.

In a case where the first compound is a compound represented by the general formula (1), the polymer of the compound represented by the general formula (1) is, for example, a homopolymer of the fluorine-containing epoxy compound represented by general formula (1), or a polymer of the fluorine-containing epoxy compound represented by general formula (1) and a monomer different from the fluorine-containing epoxy compound. Such a polymer preferably includes a structural unit derived from the compound represented by the general formula (1), and preferably represented by general formula (2).

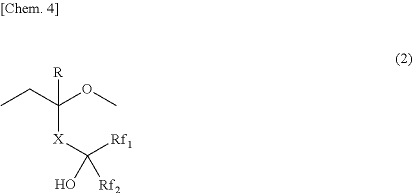

In the general formula (2), the definitions of R, X, Rf₁ and Rf₂ are the same as those in the general formula (1).

The polymerizable composition of the present embodiment may include only the first compound (preferably the compound represented by the general formula (1)) as a monomer, and may include a compound capable of reacting with the first compound (in the present specification, referred to as a “second compound”). In the former case, the polymerization reaction may be a homopolymerization reaction of the first compound, and in the latter case, the polymerization reaction may be a copolymerization reaction of the first compound and the second compound. Therefore, the obtained cured product may be a polymer including only a structural unit derived from the first compound, or a polymer including a structural unit derived from the first compound and a structural unit derived from the second compound.

In the preferable embodiment, the polymerizable composition includes the second compound. The cured product obtained by the curing treatment of the polymerizable composition includes a polymer obtained by the polymerization reaction between the first compound and the second compound. This makes it possible to obtain a cured product having excellent moldability and adhesion to a base material.

The second compound may be a compound having a group capable of forming a covalent bond by reacting with the epoxy group of the first compound (preferably the compound represented by the general formula (1)). Examples of the group capable of forming a covalent bond by reacting with the epoxy group include an epoxy group and an oxetanyl group, and among these, an epoxy group is preferable.

Examples of the compound having the group capable of forming a covalent bond by reacting with the epoxy group of the first compound include a monofunctional epoxy compound having one epoxy group and a polyfunctional epoxy compound having two or more epoxy groups.

Examples of the monofunctional epoxy compound include 4-tert-butylphenyl glycidyl ether, m,p-cresyl glycidyl ether, phenyl glycidyl ether, and cresyl glycidyl ether.

Examples of the polyfunctional epoxy compound include polyglycidyl ethers such as ethylene glycol diglycidyl ether, polyethylene glycol diglycidyl ether, polypropylene glycol diglycidyl ether, glycerin polyglycidyl ether, glycerol polyglycidyl ether, diglycerol polyglycidyl ether, polyglycerol polyglycidyl ether, trimethylolpropane polyglycidyl ether, 1,6-hexanediol diglycidyl ether, sorbitol polyglycidyl ether, pentaerythritol polyglycidyl ether, resorcinol diglycidyl ether, neopentyl glycol diglycidyl ether, and hydrogenated bisphenol A type diglycidyl ether.

Of course, the second compound is not limited only to these compounds.

As the compound having the group capable of forming a covalent bond by reacting with the epoxy group of the first compound, compounds such as an alicyclic epoxy compound, a polymer having an epoxy structure, a novolac type epoxy resin, and a siloxane-based monomer having an epoxy structure can also be used.

Examples of the alicyclic epoxy compound include 1,2-epoxy-4-vinylcyclohexane (trade name: CELLOXIDE 2000, manufactured by Daicel Corporation), which is a monofunctional epoxy, and 3′,4′-epoxycyclohexylmethyl-3,4-epoxycyclohexanecarboxylate (trade name: CELLOXIDE 2021P, manufactured by Daicel Corporation), which is a polyfunctional epoxy.

Examples of the polymer having an epoxy structure include a polymer obtained by polymerizing or copolymerizing a (meth)acrylate-based monomer having an epoxy structure. Examples of the (meth)acrylate-based monomer having an epoxy structure include glycidyl methacrylate, 4-hydroxybutyl acrylate glycidyl ether (abbreviation: 4HBAGE, manufactured by Mitsubishi Chemical Corporation), and 3,4-epoxycyclohexyl methyl methacrylate (trade name: CYCLOMER M100, manufactured by Daicel Corporation), which are monofunctional epoxies.

Examples of the siloxane-based monomer having an epoxy structure include 2-(3,4-epoxycyclohexyl)ethyltrimethoxysilane (trade name: KBM-303, manufactured by Shin-Etsu Chemical Co., Ltd.), and 3-glycidoxypropyltrimethoxysilane (trade name: KBM-403, manufactured by Shin-Etsu Chemical Co., Ltd.), which are monofunctional epoxies.

Examples of the novolac type epoxy resin include jER 152 (manufactured by Mitsubishi Chemical Corporation), EPICLON N730-A (manufactured by DIC Corporation), and YDPN-638 (manufactured by NIPPON STEEL Chemical & Material Co., Ltd.).

When the polymerizable composition of the present embodiment includes the second compound, the amount thereof is, for example, 20% by mass or more and 80% by mass or less, and preferably 30% by mass or more and 70% by mass or less, with respect to the total solid content of the polymerizable composition.

As another viewpoint, the amount of the reactive compound is, for example, 40% by mass or more and 80% by mass or less, and preferably 50% by mass or more and 70% by mass or less, with respect to the first compound included in the polymerizable composition.

The polymerizable composition of the present embodiment preferably includes a cationic polymerization initiator in order to promote polymerization. Examples of the cationic polymerization initiator include a photocationic polymerization initiator that generates an acid by light. By using such a photocationic polymerization initiator, the polymerizable composition of the present embodiment can be patterned by a photolithography treatment.

Examples of the cationic polymerization initiator include azo compounds such as azobisisobutyronitrile (AIBN), dimethyl 2,2′-azobis(2-methylpropionate), and 1,1′-azobis(cyclohexanecarbonitrile) (ABCN), sulfonium salts such as triphenylsulfonium trifluoromethanesulfonate, and tris(4-t-butylphenyl)sulfonium-trifluoromethanesulfonate; diazonium salts such as p-nitrophenyldiazonium hexafluorophosphate; ammonium salts; phosphonium salts; iodonium salts such as diphenyliodonium trifluoromethane sulfonate, and (tricumyl)iodonium-tetrakis(pentafluorophenyl)borate; quinone diazides; diazomethanes such as bis(phenylsulfonyl)diazomethane; sulfonate esters such as 1-phenyl-1-(4-methylphenyl)sulfonyloxy-1-benzylmethane, and N-hydroxynaphthallimide-trifluoromethane sulfonate; disulfones such as diphenyldisulfone; and triazines such as tris(2,4,6-trichloromethyl)-s-triazine, and 2-(3,4-methylenedioxyphenyl)-4,6-bis(trichloromethyl)-s-triazine. However, there is no limitation thereto.

In a case where the polymerizable composition of the present embodiment includes a cationic polymerization initiator, the amount thereof is, for example, 0.1% by mass or more and 10.0% by mass or less, preferably 0.2% by mass or more and 5.0% by mass or less, and more preferably 0.5% by mass or more and 4.0% by mass or less, with respect to the total solid content of the polymerizable composition. By using the cationic polymerization initiator in an amount within the above range, a cured product having excellent moldability can be obtained.

The polymerizable composition of the present embodiment may include additives such as a solvent, a photosensitive agent, a plasticizer, a coupling agent, a surfactant, an adhesion aid, a sensitizer, and a filler, depending on the characteristics desired for the application thereof.

<Cured Product, Electronic Device, and Optical Member>

The cured product of the curable composition according to the present embodiment includes a polymer of the first compound (preferably the compound represented by the general formula (1)).

Since this cured product has excellent base material adhesion and a low refractive index property, for example, the cured product is used as a resin film such as a permanent film for electronic devices, and an optical film for optical members. That is, the electronic device provided with the cured product of the curable composition has good reliability due to the excellent base material adhesion of the cured product. In addition, the optical member provided with the cured product of the curable composition has good optical properties.

The resin film formed of the cured product of the present embodiment is prepared by applying the above-mentioned curable composition to a base material to obtain a coating film and removing the solvent. The coating method is not particularly limited, and examples thereof include spin coating, roll coating, flow coating, dip coating, spray coating, and doctor coating. The base material (substrate) to which the composition is applied is not particularly limited. Examples thereof include a glass substrate, a silicon wafer, a ceramic substrate, an aluminum substrate, a SiC wafer, a GaN wafer, and a copper clad laminate. A circuit, an element, or any other layer may or may not be formed on the base material (substrate).

The curable composition is mainly cured by photocuring. Curing is carried out by, for example, light irradiation at a wavelength of 365 nm and in an exposure amount of 3000 to 30000 mJ/cm². The light source is preferably an LED, a high-pressure mercury lamp, a metal halide lamp, or the like.

The polymerizable composition applied on the base material may be cured by a heating treatment. Heating is typically performed using a hot plate, hot air, an oven, or the like. The heating temperature is usually 50° C. to 140° C., preferably 70° C. to 90° C. in consideration of damage to the device. The heating time is usually about 30 to 600 seconds, and preferably about 30 to 300 seconds.

The film thickness of the coating film is not particularly limited and may vary depending on the application. The film thickness is usually 0.5 to 10 μm, and preferably 1 to 5 μm. The film thickness can be adjusted by adjusting the content of the solvent in the polymerizable composition, the coating method, and the like.

In a case where the resin film of the present embodiment is used as, for example, a permanent film for an electronic device, the coating film of the resin composition obtained as described above is exposed and developed, patterned into a desired shape, and then cured by a heat treatment or the like to obtain the resin film.

<Application of First Compound to Photocurable Adhesive>

As an application example of the first compound, an application to a photocurable adhesive will be described below.

Development of display devices such as various displays, touch panels, and smartphones has been continuously performed. Along with this, various adhesives capable of forming a cured film having light transmittance have been studied. Such an adhesive is used, for example, in a display device having a multi-layer structure for joining between layers.

Particularly, recently, a display having a foldable screen called a “foldable display” has been actively studied. In addition, a smartphone equipped with a foldable display, that is, a foldable smartphone, has been actively developed.

Therefore, there is a growing demand for an adhesive suitable for producing a foldable display.

Since a foldable display is a kind of display device, the adhesive applied to the production of the foldable display is required to have the same characteristics as the adhesive applied to the production of the conventional display device. Specifically, the required characteristics include a small difference in the refractive index between a base material such as glass or a resin film and a cured film, and good adhesion to a base material such as glass or a resin film.

Further, in a case where the adhesive is photocurable, the adhesive is required to have appropriate photocurability.

In addition, the cured film formed of the adhesive applied to the production of a foldable display is required to have “bending resistance”. Specifically, even when the cured film is bent, it is required that cracks and wrinkles are less likely to occur.

The present inventors have found that a curable composition (photocurable adhesive) including (i) the above-mentioned first compound, (ii) a photocationic polymerization initiator, and (iii) a compound (P) which has an aromatic ring skeleton and/or an alicyclic skeleton and in which the total of the number of hydroxy groups and the number of epoxy groups per molecule is 2 or more is suitable for producing a foldable display.

Although speculated, the cured product of the above-mentioned curable composition (photocurable adhesive) has a relatively low refractive index since the first compound includes a fluorine atom (including a fluorine-containing alkyl group).

In addition, since the first compound includes an epoxy group, it is considered that the cured product of the above-mentioned curable composition (photocurable adhesive) exhibits good adhesiveness to a base material (typically glass or PET film) used in the production of a display device.

Further, since the first compound includes an oxygen atom (hydroxy group) adjacent to the fluorinated alkyl group, it is considered that the cured product of the above-mentioned curable composition (photocurable adhesive) becomes relatively flexible and the bending resistance is enhanced. Although the details are unknown, it is considered that since the cured product of the photocurable adhesive of the present embodiment has a structure derived from the fluorinated alkyl group and the oxygen atom (hydroxy group), the interaction between the polymers in the cured product is reduced (it is presumed that the large electron withdrawing properties of the fluorinated alkyl group and the like are involved). It is considered that this reduced interaction leads to the flexibility of the cured product and eventually, the bending resistance.

In addition, it is considered that the hydroxy group and the epoxy group of the compound (P) contribute to appropriate photocurability and adhesion to the base material.

In addition, it is considered that the aromatic ring skeleton and/or the alicyclic skeleton included in the compound (P) contributes to the promotion of curing and the improvement of the mechanical properties of the cured film.

The compound (P) preferably has an aromatic ring skeleton or a polycyclic alicyclic skeleton from the viewpoint of the rigidity of the cured film or the like.

As the compound (P), an epoxy resin can be preferably exemplified. That is, an epoxy resin having an aromatic ring skeleton and/or an alicyclic skeleton can be exemplified as a preferable compound (P).

More specifically, it is preferable that the compound (P) includes one or more epoxy resins selected from the group consisting of a bisphenol A type epoxy resin, a bisphenol F type epoxy resin, a dicyclopentadiene type epoxy resin, a naphthalene type epoxy resin, a cresol novolac type epoxy resin, and a phenol novolac type epoxy resin. By using any of these epoxy resins, the balance of various performances can be enhanced.

In addition, as an example of the compound (P) having a hydroxy group, a compound in which the glycidyl group is replaced with a hydroxy group in the above-mentioned epoxy resin can be mentioned. That is, a bisphenol A type phenol resin, a bisphenol F type phenol resin, a novolac resin, a resole resin and the like can be exemplified. Incidentally, the hydroxy group that the compound (P) can have may be an alcoholic hydroxy group or a phenolic hydroxy group.

The above-mentioned curable composition (photocurable adhesive) may include only one compound (P) or may include two or more compounds (P).

The amount of the compound (P) used is preferably 10% to 80% by mass, and more preferably 20% to 70% by mass in the non-volatile component of the curable composition (photocurable adhesive).

Specific examples of the first compound that can be preferably used in the above-mentioned curable composition (photocurable adhesive) are as described above.

By appropriately adjusting the amount of the first compound, performance such as bending resistance can be further enhanced.

Specifically, it is preferable that the amount of the first compound is adjusted so that the number of moles of the first compound included in 100 g of a non-volatile component of the curable composition (photocurable adhesive) is 0.04 to 0.4 mol. The number of moles is more preferably 0.05 to 0.4 mol, and even more preferably 0.1 to 0.3 mol.

The above-mentioned curable composition (photocurable adhesive) may include only one first compound, or two or more first compounds having different structures.

The amount of the first compound used is preferably 10% to 90% by mass, and more preferably 20% to 80% by mass in the non-volatile component of the above-mentioned curable composition (photocurable adhesive).

Examples of the photocationic polymerization initiator that can be preferably used in the above-mentioned curable composition (photocurable adhesive) include those described above, and particularly, an onium salt compound can be preferably exemplified.

Specific examples thereof include photoacid generators or photocationic initiators such as diazonium salts, iodonium salts such as diaryliodonium salts, sulfonium salts such as triarylsulfonium salts, triarylpyrylium salts, benzylpyridinium thiocyanate, dialkylphenacylsulfonium salts, and dialkylhydroxyphenylphosphonium salts.

Among these, it is preferable to use a triarylsulfonium salt from the viewpoint of sensitivity and storage stability.

Examples of counter anions of the onium salt compound include borate anions, sulfonate anions, gallate anions, phosphorus-based anions, and antimony-based anions. More specific examples thereof include sulfonate anions, disulfonylimide acid anions, hexafluorophosphate anions, fluoroantimonate anions, tetrafluoroborate anions, and tetrakis(pentafluorophenyl)borate anions.

The above-mentioned curable composition (photocurable adhesive) may include only one photocationic polymerization initiator, or may include two or more different photocationic polymerization initiators.

The amount of the photocationic polymerization initiator used is, for example, 0.1% to 5.0% by mass, preferably 0.2% to 4.5% by mass, and more preferably 0.5% to 4.0% by mass, in the non-volatile component of the above-mentioned curable composition (photocurable adhesive).

The above-mentioned curable composition (photocurable adhesive) may also include various other optional components. For example, the alicyclic epoxy compounds exemplified in the above-described “second compound”, and among above examples, particularly, a polyfunctional alicyclic epoxy compound can be exemplified as an optional component that can be preferably used.

Examples of other optional components include organic solvents, plasticizers, coupling agents, surfactants, adhesion aids, sensitizers, and fillers.

However, it is preferable that the above-mentioned curable composition (photocurable adhesive) does not include an organic solvent. Even in a case where the curable composition includes an organic solvent, it is preferable that the amount of the organic solvent is small. This is because it is not necessary to provide a heating step or a drying step for volatilizing the organic solvent. In the above-mentioned curable composition (photocurable adhesive), since the first compound can function as a reactive diluent, a curable composition (photocurable adhesive) capable of forming a coating film without using an organic solvent can be prepared.

Specifically, the content of the organic solvent in the photocurable adhesive of the present embodiment is preferably 0% to 10% by mass, more preferably 0% to 5% by mass, and even more preferably 0% to 1% by mass. It is particularly preferable that the photocurable adhesive of the present embodiment does not substantially include the organic solvent. However, the photocurable adhesive of the present embodiment does not exclude impurities in the raw material and the organic solvent inevitably included due to the production atmosphere.

The viscosity of the above-mentioned curable composition (photocurable adhesive) is preferably 10 to 10000 mPa·s, and more preferably 100 to 6000 mPa·s from the viewpoint of good coatability, film forming properties, and the like. The viscosity can be adjusted by appropriately selecting the material of each component and the amount thereof.

The viscosity is preferably measured at a shear rate of 100 (1/s) using a rheometer.

The cured film, which is a cured product obtained by irradiating the above-mentioned curable composition (photocurable adhesive) with light, is usually sufficiently transparent. Specifically, the light transmittance of the cured film having a film thickness of 100 μm, obtained by curing the above-mentioned curable composition (photocurable adhesive), at a wavelength of 400 nm is preferably 90% or more, and more preferably 95% or more. Basically, the larger the light transmittance is, the better it is, but the upper limit value of the light transmittance is practically 99%.

For examples of the conditions for forming the cured film, examples described later are referred to.

The above-mentioned curable composition (photocurable adhesive) is typically applied onto glass or a resin film (preferably a polyester film such as PET) to form a coating film, and the coating film is exposed (irradiated with active rays such as ultraviolet rays), so that a semi-cured film or a cured film can be obtained.

Examples of the coating method include bar coating, spin coating, roll coating, flow coating, dip coating, spray coating, and doctor coating. After coating, prebaking may be performed.

Exposure is carried out by, for example, light irradiation at a wavelength of 365 nm and in an exposure amount of 3000 to 30000 mJ/cm². Examples of the light source include an LED, a high-pressure mercury lamp, and a metal halide lamp.

Although the above-mentioned curable composition can be completely cured only by exposure, a heat treatment may be performed in addition to exposure to more completely cure the curable composition. Heating is typically performed using a hot plate, hot air, an oven or the like. The heating temperature is usually 50° C. to 140° C., and preferably 70° C. to 90° C. in consideration of damage to the base material. The heating time is usually about 30 to 600 seconds, and preferably about 30 to 300 seconds.

The thickness of the cured film is typically about 50 to 200 μm, and is appropriately adjusted depending on the structure of the display device.

As described above, by using the above-mentioned curable composition (photocurable adhesive), a display device such as a foldable display can be preferably produced. That is, a display device having a multi-layer structure can be produced by laminating a plurality of base materials such as a glass base material or a polyester film (such as a PET film) with the curable composition (photocurable adhesive).

Just for the description, the application of the above-mentioned curable composition (photocurable adhesive) is not limited to the production of a foldable display. It is considered that the application of the curable composition is not limited to the production of a foldable display and the curable composition can be applied to, for example, the production of a rollable display (a display that can be “rolled”) and a stretchable display.

An example of the structure of the foldable display is described in, for example, EP3644383A. FIG. 4 in EP3644383A shows a schematic cross-sectional view of a foldable display in a folded state. In FIG. 4, adhesive films 901, 911, and 921 are included. The above-mentioned curable composition (photocurable adhesive) is preferably used for forming these adhesive films.

Particularly, the adhesive film of reference numeral 921 has a large curvature in the folded state of the foldable display. Since the cured product of the above-mentioned curable composition (photocurable adhesive) has good bending resistance, the above-mentioned curable composition (photocurable adhesive) can be preferably applied to the formation of the adhesive film of reference numeral 921.

In the production of a foldable display, it is preferable to perform a process of laminating a base material while bending a photocurable adhesive in a semi-cured state as in the following step example. By doing so, it is possible to add an appropriate “crease” to the bent portion of the foldable display.

### Step Example

(1) A coating film of the photocurable adhesive is formed on the surface of a base material.

(2) The coating film is semi-cured by exposure.

(3) While bending the semi-cured coating film, another base material (cover material: ultra-thin glass, a polyester film, a transparent polyimide film, or the like) is attached to the coating film.

(4) The semi-cured coating film is completely cured by exposure.

A supplementary note about “semi-curing” will be given.

Semi-curing refers to a state in which a certain base material can be attached to and peeled off from the film after exposure, and the film is completely cured by applying additional exposure and/or heating to the semi-cured film and peeled off. The above-mentioned curable composition (photocurable adhesive) can be semi-cured at an exposure amount of 20 to 60, when the exposure amount required for complete curing is usually 100.

Although the exposure condition varies depending on the material design, for example, the materials of the examples described later can be semi-cured under an exposure condition of about 3000 to 8000 mJ/cm².

Since the curable composition (photocurable adhesive) can be semi-cured, for example, rework can be carried out. Although it is difficult to remove the completely cured product, the semi-curable curable composition (photocurable adhesive) can be relatively easily removed. For example, removal can be carried out with an alkaline aqueous solution (tetramethylammonium hydroxide aqueous solution, potassium hydroxide aqueous solution, or the like). In addition, removal can also be carried out with an organic solvent such as tetrahydrofuran or propylene glycol monomethyl ether acetate.

<Additional Description Relating to Polymer>

A polymer including a structural unit represented by the general formula (2) and a structural unit represented by general formula (3) is preferably applicable to a photosensitive resin composition that can be patterned by exposure. Examples of applications of the photosensitive resin composition include a low refraction patterning material, and a liquid-repellent bank material. Particularly, in a case where the photosensitive resin composition is used as a low refraction patterning material, an OLED light extraction layer (waveguide), a protective layer on the upper portion of a high refractive microlens layer, and the like can be considered as the applications of the cured film having a pattern shape obtained by patterning.

In addition, it is considered that this polymer itself can be used as a coating material such as a topcoat material in semiconductor lithography.

In this polymer, it is considered that the —C(Rf₁)(Rf₂)—OH group of the structural unit represented by the general formula (2) contributes to the developability with an alkaline aqueous solution, and the structural unit represented by general formula (3) contributes to photocurability.

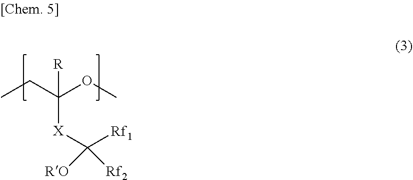

In the general formula (3),

R′ represents a monovalent organic group, and

the definitions of X, Rf₁, and Rf₂ are the same as those in the general formula (2).

In terms of negative pattern formation, the monovalent organic group of R′ preferably includes a polymerizable group. Specifically, R′ may be a monovalent organic group having a polymerizable carbon-carbon double bond. More specifically, R′ may be a monovalent organic group including at least one selected from the group consisting of a (meth)acryloyl group and a vinyl group.

As a specific embodiment of R′, chemical structures such as structural units represented by general formulas (r1) to (r3) can be exemplified.

—O(CO)C(R²)═CH₂ (r1)

—CH₂CH(OH)—R¹—O(CO)C(R²)═CH₂ (r2)

—CO—NH—R¹—O(CO)C(R²)═CH₂ (r3)

In the above formulas, R¹ represents a divalent linking group, and R² represents a hydrogen atom, a methyl group or a trifluoromethyl group.

Specific examples of the divalent linking group of R¹ include a linear, branched or cyclic alkylene group, an arylene group, —O—, —S—, —CO—, —COO—, —SO—, —SO₂—, and a group in which two or more of these groups are linked to each other.

From the viewpoint of the balance between developability and curability, the ratio of the structural unit (having a —C(Rf₁)(Rf₂)—OH group) represented by the general formula (2) is, for example, 30 to 100 mol %, preferably 30 to 90 mol %, and more preferably 30 to 80 mol %, in all the structural units of the polymer.

From the viewpoint of application to an optical member and the like, and from the viewpoint of making the finally obtained film (cured film, or the like) have a low refractive index, the ratio of fluorine atoms in the polymer is preferably 20% to 60% by mass. By setting the ratio of fluorine atoms within this range, the refractive index of the film is easily lowered without excessively impairing other performance.

The weight average molecular weight of the polymer is usually 3,000 to 30,000, and preferably 5,000 to 20,000.

The dispersity of the polymer is usually 1.2 to 3.0, and preferably 1.3 to 2.5.

Usually, a photosensitive resin composition is formed by mixing the polymer including the structural unit represented by the general formula (2) and the structural unit represented by the general formula (3), a photopolymerization initiator, and a solvent.

Particularly, in a case where the monovalent organic group of R′ in the general formula (3) is a monovalent organic group having a polymerizable carbon-carbon double bond, it is preferable that the photopolymerization initiator includes a photoradical polymerization initiator.

Specific examples of the photoradical polymerization initiator include α-hydroxyketone photoinitiators, α-aminoketone photoinitiators, bisacylphosphine photoinitiators, monoacylphosphine oxide, and bisacylphosphine oxides, for example, 2,4,6-trimethylbenzoylbiphenylphosphine oxides, ethyl-2,4,6-trimethylbenzoylphenylphosphinate, mono- and bis-acylphosphine photoinitiators, benzyldimethyl-ketal photoinitiators, and oligo[2-hydroxy-2-methyl-1-[4-(1-methylvinyl)phenyl]propanone].

Examples of commercially available photopolymerization initiators include the IRGACURE (registered trademark) series commercially available from BASF. Of course, photopolymerization initiators other than these can also be used.

In a case where the photopolymerization initiator is used, only one type may be used, or two or more types thereof may be used in combination.

In a case where the photopolymerization initiator is used, the amount thereof is usually 0.5 to 15 parts by mass, and preferably 1.0 to 10 parts by mass, with respect to 100 parts by mass of the polymer.

As the solvent, an organic solvent is preferably used. Examples of organic solvents that can be preferably used include propylene glycol monomethyl ether acetate, propylene glycol monomethyl ether, cyclohexanone, ethyl lactate, γ-butyrolactone, diacetone alcohol, diglyme, methyl isobutyl ketone, 3-methoxybutyl acetate, 2-heptanone, N,N-dimethylformamide, N,N-dimethylacetamide, and N-methylpyrrolidone.

In addition, glycols, glycol ethers, glycol ether esters and the like can also be exemplified as usable solvents. Specific examples thereof include CELTOR (registered trademark) manufactured by Daicel Corporation and HISOLVE (registered trademark) manufactured by Toho Chemical Industry Co., Ltd. More specific examples thereof include cyclohexanol acetate, dipropylene glycol dimethyl ether, propylene glycol diacetate, dipropylene glycol methyl-n-propyl ether, dipropylene glycol methyl ether acetate, 1,4-butanediol diacetate, 1,3-butylene glycol diacetate, 1,6-hexanediol diacetate, 3-methoxybutyl acetate, ethylene glycol monobutyl ether acetate, diethylene glycol monoethyl ether acetate, diethylene glycol monobutyl ether acetate, triacetin, 1,3-butylene glycol, propylene glycol-n-propyl ether, propylene glycol-n-butyl ether, dipropylene glycol methyl ether, dipropylene glycol ethyl ether, dipropylene glycol-n-propyl ether, dipropylene glycol-n-butyl ether, tripropylene glycol methyl ether, tripropylene glycol-n-butyl ether, triethylene glycol dimethyl ether, diethylene glycol butyl methyl ether, tripropylene glycol dimethyl ether, and triethylene glycol dimethyl ether.

In a case where the organic solvent is used, the amount used is not particularly limited, but the organic solvent is used so that the total solid content (components other than the organic solvent) in the resin composition is usually 5% to 60% by mass, and preferably 10% to 50% by mass. By appropriately adjusting the total solid content concentration, the ease of forming a thin film and the uniformity of the film thickness tend to be improved.

The photosensitive resin composition may also include one or more of a cross-linking agent, a polymerization inhibitor, an ultraviolet absorber, a chain transfer agent, and the like.

The polymer including the structural unit represented by the general formula (2) and the structural unit represented by the general formula (3) may be produced by any of the following methods.

(i) First, a polymer which includes the structural unit represented by the general formula (2) but does not include the structural unit represented by the general formula (3) is prepared. Then, the —OH moiety in the polymer is modified.

(ii) The compound represented by the general formula (1) and a compound in which R in the compound represented by the general formula (1) is R′ (the definition of R′ is as described above) are copolymerized.

Incidentally, regarding the introduction of the structure of the general formula (r1) above, the description of (Synthesis of BTHB-epo-A) in <Additional Examples Relating to Polymers> described later is also referred to.

Regarding the introduction of the structure of the general formula (r2), the description of [Polymerization of BTHB-epo-Containing Fluororesin 3] in <Additional Examples Relating to Polymers> described later is also referred to (the structure of the general formula (r2) is introduced using an epoxy group-containing compound).

Regarding the introduction of the structure of the general formula (r3), the description of [Polymerization of BTHB-epo-Containing Fluororesin 4] in <Additional Examples Relating to Polymers> described below is also referred to (the structure of the general formula (r3) is introduced using an isocyanate group-containing compound).

Although the embodiments of the present invention have been described above, these are examples of the present invention, and various compositions other than the above can be adopted.

### EXAMPLES

Hereinafter, the present invention will be described with reference to Examples and Comparative Examples, but the present invention is not limited thereto.

### Synthesis Example A1

Synthesis of Fluorine-Containing Epoxy Compound A1 (BTHB-epo)

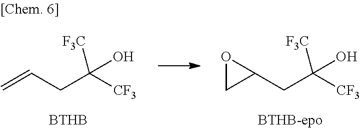

In a 3 L three-necked eggplant flask, BTHB (166.46 g, 800 mmol) (manufactured by Central Glass Co., Ltd.) and dichloromethane (800 ml) were mixed, and ice-cooled, and mCPBA (195.26 g, 792 mmol) was added thereto in small portions. Then, after stirring overnight at 35° C., the mixture was washed with a 10 wt % sodium thiosulfate aqueous solution once and was washed with a 5 wt % sodium bicarbonate aqueous solution once, and the resultant was concentrated by an evaporator. The concentrated solution was distilled under reduced pressure (recovered at 2 kPa and 67° C. to 70° C.) to obtain BTHB-epo (146.9 g, yield: 82%).

¹H-NMR (CDCl₃): 4.30 ppm (s, 1H, OH group), 3.32 ppm (br, 1H), 2.93 ppm (t, 4.4 Hz, 1H), 2.60 ppm (dd, 4.4 Hz), 2.4 Hz, 1H), 2.47 ppm (dd, 15.2 Hz, 3.6 Hz, 1H), 1.86 ppm (ddd, 15.2 Hz, 8.8 Hz, 1.6 Hz, 1H)

¹⁹F-NMR: −78.5 ppm (q, 12.4 Hz, 6F)

### Synthesis Example A2

Synthesis of Fluorine-Containing Epoxy Compound A2 (Me-BTHB-epo)

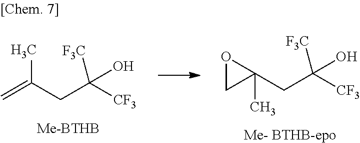

Using the same method as in Synthesis Example 1, the concentrated solution was distilled under reduced pressure (recovered at 1.3 kPa and 70° C. to 72° C.) to obtain Me-BTHB-epo (67 g, yield: 78%).

¹H-NMR (CDCl₃): 4.13 ppm (s, 1H, OH group), 2.91 ppm (d, 4.2 Hz, 1H), 2.65 ppm (d, 4.2 Hz, 1H), 2.49 ppm (dd, 14.1 Hz, 3.4 Hz, 1H), 1.81 ppm (dd, 14.1 Hz, 3.4 Hz, 1H), 1.57 ppm (s, 3H, methyl group)

¹⁹F-NMR: −78.3 ppm (q, 12.4 Hz, 6F)

### Synthesis Example A3

Synthesis of Fluorine-Containing Epoxy Compound A3 (MeO-BTHB-epo)

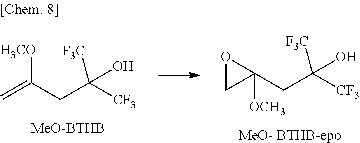

Using the same method as in Synthesis Example 1, the concentrated solution was distilled under reduced pressure (recovered at 1.7 kPa and 75° C. to 78° C.) to obtain Me-BTHB-epo (33 g, yield: 72%).

¹H-NMR (CDCl₃): 4.01 ppm (s, 1H, OH group), 3.80 ppm (s, 3H, methoxy group), 2.83 ppm (d, 4.5 Hz, 1H), 2.55 ppm (d, 4.5 Hz, 1H), 2.44 ppm (dd, 15.0 Hz, 3.0 Hz, 1H), 1.98 ppm (dd, 15.0 Hz, 3.0 Hz, 1H)

¹⁹F-NMR: −78.5 ppm (q, 12.4 Hz, 6F)

The storage stability of the fluorine-containing epoxy compound A1 (BTHB-epo) obtained above was evaluated. For storage stability, the amount of a ring-opened body formed by ring-opening of the epoxy group of the fluorine-containing epoxy compound was measured immediately after purification, after 1 week, after 2 weeks, after 3 weeks, and after 1 month. At each time point, evaluation was performed by measurement by 19F-NMR. The amount of the ring-opened body was calculated by using the increase amount in peaks other than BTHB-epo in the 19F-NMR chart as the ring-opened body. The smaller the amount of the ring-opened body, the better the storage stability. The results are shown in Table 1 below.

### [Table 1]

TABLE 1

| | | Liquid | Amount of |
|---|---|---|---|
| | Viscosity | temperature | ring-opened body |
| Storage period | (mPa · s) | (° C.) | 19F-NMR (mol %) |
| Immediately | 15.45 | 24.8 | 0.03 |
| after | | | |
| purification | | | |
| 1 Week | 15.65 | 24.3 | 0.05 |
| 2 Weeks | 15.60 | 24.9 | 0.06 |
| 3 Weeks | 15.55 | 24.7 | 0.06 |
| 1 Month | 15.60 | 24.6 | 0.07 |

### Synthesis Example B1

Synthesis of Polymer 1 (HFIP-M/4HBAGE=70/30 (corresponding to polyfunctional epoxy compound))

17.4 g of hexafluoroisopropyl methacrylate (HFIP-M), 5.3 g of 4-hydroxybutyl acrylate glycidyl ether (4HBAGE), and 0.41 g of dimethyl 2,2′-azobis(2-methylpropionate) (V-601) were dissolved in 40 g of methyl ethyl ketone and were allowed to react at 80° C. for 6 hours.

The reaction solution was concentrated and reprecipitated with heptane to obtain 15.4 g of polymer 1. The Mw of the obtained polymer 1 was 10,600, and the Mw/Mn was 2.74.

### Synthesis Example B2

Synthesis of Polymer 2 (OFP-M/4HBAGE=70/30 (corresponding to polyfunctional epoxy compound))

15.4 g of octafluoropropyl methacrylate (OFP-M), 4.61 g of 4-hydroxybutyl acrylate glycidyl ether (4HBAGE), and 0.35 g of dimethyl 2,2′-azobis(2-methylpropionate) (V-601) were dissolved in 40 g of methyl ethyl ketone and were allowed to react at 80° C. for 6 hours.

The reaction solution was concentrated and reprecipitated with heptane to obtain 13.8 g of polymer 2. The Mw of the obtained polymer 2 was 26,600, and the Mw/Mn was 1.80.

### Synthesis Example B3

Synthesis of Polymer 3 (MA-BTHB-OH Homopolymer)

20 g of 5,5,5-trifluoro-4-hydroxy-4-(trifluoromethyl)-2-pentyl-2-methacrylate (MA-BTHB-OH), and 0.30 g of dimethyl 2,2′-azobis (2-methylpropionate) (V-601) were dissolved in 40 g of methyl ethyl ketone and were allowed to react at 80° C. for 6 hours.

The reaction solution was concentrated and reprecipitated with heptane to obtain 16.0 g of polymer 3. The Mw of the obtained polymer 3 was 17,000, and the Mw/Mn was 1.95.

### Synthesis Example B4

Synthesis of Polymer 4 (HFIP-M/MA-BTHB-OH=40/60)

7.0 g of hexafluoroisopropyl methacrylate (HFIP-M), 13.0 g of MA-BTHB-OH, and 0.34 g of dimethyl 2,2′-azobis(2-methylpropionate) (V-601) were dissolved in 40 g of methyl ethyl ketone and were allowed to react at 80° C. for 6 hours.

The reaction solution was concentrated and reprecipitated with heptane to obtain 17.3 g of polymer 4. The Mw of the obtained polymer 4 was 15,500, and the Mw/Mn was 1.88.

The weight average molecular weight Mw and the dispersity Mw/Mn of the polymer were measured by GPC measurement under the following conditions.

GPC measurement condition: Using HLC-8320 GPC manufactured by Tosoh Corporation, measurement was performed under the condition in which tetrahydrofuran (THF) was allowed to flow through a column at a flow rate of 1 mL/min as an elution solvent to perform elution. The values of the weight average molecular weight and the dispersity are calculated using polystyrene as a standard substance.

### Examples 1 to 3 and Comparative Examples 3 to 5

According to the composition shown in Table 2 below, component (A): a monomer (first compound), component (B): a polyfunctional compound (second compound), and the component (C): a polymerization initiator were dissolved at a predetermined ratio, and stirred at room temperature for 3 hours to obtain a uniform solution. Thus, an ultraviolet curable composition was prepared.

### [Table 2]

TABLE 2

| | Component (A) | Component (B) | Component (C) |
|---|---|---|---|
| Example 1 | BTHB-ep | Polymer 1 | CPI-210S |
| | 0.7 g | 0.3 g | 0.01 g |
| Example 2 | BTHB-ep | Polymer 2 | CPI-210S |
| | 0.7 g | 0.3 g | 0.01 g |
| Example 3 | BTHB-ep | CELLOXIDE 202IP | CPI-210S |
| | 0.7 g | 0.3 g | 0.02 g |
| Comparative | Polymer 3 | — | — |
| Example 1 | (MA-BTHB-OH | | |
| | homopolymer) | | |
| Comparative | Polymer 4 | — | — |
| Example 2 | (HFIP-M/MA-BTHB-OH) | | |
| Comparative | MA-BTHB-OH (monomer) | ARONIX M-310 | IRGACURE |
| Example 3 | 0.7 g | 0.3 g | 1173 |
| | | | 0.02 g |
| Comparative | MA-BTHB-OH (monomer) | Pentaerythritol | IRGACURE |
| Example 4 | 0.7 g | Tetraacrylate | 1173 |
| | | 0.3 g | 0.02 g |
| Comparative | MA-BTHB-OH (monomer) | NK Ester A-9550 | IRGACURE |
| Example 5 | 0.7 g | 0.3 g | 1173 |
| | | | 0.02 g |

The components listed in Table 2 are as follows.
CPI-201S: Photoacid generator (photocation generator, that is, cationic polymerization initiator), manufactured by San-Apro Ltd.
IRGACURE 1173: Photoradical initiator, manufactured by BASF CELLOXIDE 2021P: 3′,4′-Epoxycyclohexylmethyl-3,4-epoxycyclohexanecarboxylate, manufactured by Daicel Chemical Industries, Ltd.
ARONIX M-310: Trimethylolpropane PO-modified triacrylate, manufactured by Toagosei Co., Ltd.
Pentaerythritol Tetraacrylate: Pentaerythritol tetrakis acrylate, manufactured by Tokyo Chemical Industry Co., Ltd.
NK Ester A-9550: Dipentaerythritol polyacrylate, manufactured by Shin-Nakamura Chemical Co., Ltd.

### Evaluation of Adhesion of Cured Film

### Examples 1 to 3 and Comparative Examples 3 to 5

After forming a film of the obtained curable composition on various substrates (a glass substrate, a silicon wafer, a polyethylene terephthalate substrate) using a bar coater, each substrate with the film was put in a transparent vinyl bag, the transparent vinyl bag was sealed with nitrogen, and then the composition was irradiated with ultraviolet rays in an exposure amount of 15,000 mJ/cm² using a 365 nm UV-LED, to prepare a resin cured film. It is considered that this cured film includes the polymer including the structural unit represented by the general formula (2). A cross-cut test (JIS K 5600 5-6) was performed on this cured film.

### Comparative Examples 1 and 2

Using the fluorine-containing polymer 3 and the fluorine-containing polymer 4 (both are acrylic types including MA-BTHB-OH), 1 g of the polymer was dissolved in 3 g of propylene glycol monomethyl ether acetate (PGMEA) to prepare a solution, and a coating film was formed on each of the above substrates using a spin coater. A cross-cut test (JIS K 5600 5-6) was performed on this coating film.

As for the determination, a film whose cross-cut test result was classification of 0 was rated as acceptable (◯), and the others were rated as unacceptable (X).

### [Table 3]

TABLE 3

| | Adhesion evaluation | | |
|---|---|---|---|
| | To glass | To Si wafer | To PET |
| Example 1 | ◯ | ◯ | ◯ |
| Example 2 | ◯ | ◯ | ◯ |
| Example 3 | ◯ | ◯ | ◯ |
| Comparative | X | X | X |
| Example 1 | | | |
| Comparative | X | X | X |
| Example 2 | | | |
| Comparative | X | X | X |
| Example 3 | | | |
| Compara t i ve | X | X | X |
| Example 4 | | | |
| Comparative | X | X | X |
| Example 5 | | | |

As can be seen from Table 3, it was found that the cured films obtained from the compositions of Examples 1 to 3 have excellent adhesion.

### Measurement of Refractive Index

### Preparation of Ultraviolet Curable Composition

### Example 4 and Comparative Examples 6 to 8

According to the composition shown in Table 4 below, component (A): a monomer (first compound), component (B): a polyfunctional compound (second compound), and component (C): a polymerization initiator were dissolved at a predetermined ratio, and stirred at room temperature for 3 hours to obtain a uniform solution. Thus, a curable composition of each example was prepared.

### [Table 4]

TABLE 4

| | Component (A) | Component (B) | Component (C) |
|---|---|---|---|
| Example 4 | BTHB-ep | CELLOXIDE 2021P | CPI-210S |
| | 0.7 g | 0.3 g | 0.02 g |
| Comparative | n-Butyl glycidyl ether | CELLOXIDE 2021P | CPI-210S |
| Example 6 | 0.7 g | 0.3 g | 0.02 g |
| Comparative | — | CELLOXIDE 2021P | CPI-210S |
| Example 7 | | 1.0 g | 0.02 g |
| Comparative | Nonafluoropentyloxysilane | CELLOXIDE 2021P | CPI-210S |
| Example 8 | 0.7 g | 0.3 g | 0.02 g |

### Evaluation of Refractive Index of Cured Film of Ultraviolet Curable Composition

### Example 4 and Comparative Examples 6 to 8

After forming a film of the ultraviolet curable composition on a silicon substrate using a bar coater, the substrate with the film was put in a transparent vinyl bag, the transparent vinyl bag was sealed with nitrogen, and then the composition was irradiated with ultraviolet rays in an exposure amount of 15,000 mJ/cm² using a 365 nm UV-LED, to prepare a resin cured film. The refractive index of this cured film was performed by a prism coupler (2010/M, manufactured by Metricon Corporation, wavelength: 632 nm). The results are shown in Table 5.

### [Table 5]

TABLE 5

| | | Refractive index |
|---|---|---|
| | Film uniformity | (at 632 nm) |
| Example 4 | Uniform film | 1.435 |
| Comparative | Uniform film | 1.480 |
| Example 6 | | |
| Comparative | Uniform film | 1.521 |
| Example 7 | | |
| Comparative | Cloudy film | |
| Example 8 | | |

As can be seen from Table 5, it was found that the cured film prepared by using the composition of Example 4 can have a low refractive index, a uniform film can be obtained, and excellent optical properties are obtained.

<Examples Relating to Photocurable Adhesives>

Each component shown in Tables 6 and 7 below was uniformly mixed to prepare a photocurable adhesive.

Incidentally, in Tables 8 and 9 shown below, the symbols in the column of “liquid regulating properties” mean the following.

◯ (Good): It was visually confirmed that each component was mixed to form a uniform solution within 60 minutes.

Δ (Acceptable): It was visually confirmed that each component was mixed to form a uniform solution within 60 minutes or longer and within 24 hours.

X (Poor): A uniform solution was not obtained within 24 hours.

TABLE 6
Preparation of adhesive composition(Examples)

| | | | | | | Photocationic | | | |
|---|---|---|---|---|---|---|---|---|---|
| | | | | | | polymerization | | | |
| | Compound (P) | | First compound | | | initiator | | Additive | |
| | | Parts | | Number | Parts | | Parts | | Parts |
| Example | Type | by mass | Type | of moles | by mass | Type | by mass | Type | by mass |
| 5-1 | EPICLON 850 | 65 | BTHB-epo | 0.13 | 30 | CPI-210S | 1 | CELLOXIDE | 5 |
| 5-2 | EPICLON 850 | 55 | BTHB-epo | 0.18 | 40 | CPI-210S | 1 | CELLOXIDE | 5 |
| 5-3 | EPICLON 850 | 45 | BTHB-epo | 0.22 | 50 | CPI-210S | 1 | CELLOXIDE | 5 |
| 5-4 | EPICLON 850 | 35 | BTHB-epo | 0.27 | 60 | CPI-210S | 1 | CELLOXIDE | 5 |
| 5-5 | EPICLON 850 | 25 | BTHB-epo | 0.31 | 70 | CPI-210S | 1 | CELLOXIDE | 5 |
| 5-6 | EPICLON 850 | 50 | BTHB-epo | 0.22 | 50 | CPI-210S | 1 | No additives | 0 |
| 5-7 | EPICLON 830 | 45 | BTHB-epo | 0.22 | 50 | CPI-210S | 1 | CELLOXIDE | 5 |
| 5-8 | EPICLON 3050 | 45 | BTHB-epo | 0.22 | 50 | CPI-210S | 1 | CELLOXIDE | 5 |
| 5-9 | EPICLON HP7200 | 45 | BTHB-epo | 0.22 | 50 | CPI-210S | 1 | CELLOXIDE | 5 |
| 5-10 | EPICLON HP4700 | 45 | BTHB-epo | 0.22 | 50 | CPI-210S | 1 | CELLOXIDE | 5 |
| 5-11 | EPICLON N-680 | 45 | BTHB-epo | 0.22 | 50 | CPI-210S | 1 | CELLOXIDE | 5 |
| 5-12 | EPICLON N-775 | 45 | BTHB-epo | 0.22 | 50 | CPI-210S | 1 | CELLOXIDE | 5 |

TABLE 7
Preparation of adhesive composition (Comparative Examples)

| | | | | | Photocationic | | | |
|---|---|---|---|---|---|---|---|---|
| | Compound (P) | | Reactive diluent | | initiator | | Additive | |
| Comparative | | Parts | | Parts | | Parts | | Parts |
| Example | Type | by mass | Type | by mass | Type | by mass | Type | by mass |
| 9-1 | EPICLON 850 | 45 | DY-BP | 50 | CPI-210S | 1 | CELLOXIDE | 5 |
| 9-2 | EPICLON 850 | 25 | DY-BP | 70 | CPI-210S | 1 | CELLOXIDE | 5 |
| 9-3 | EPICLON 850 | 45 | FAEP-6 | 50 | CPI-210S | 1 | CELLOXIDE | 5 |
| 9-4 | EPICLON 850 | 15 | FAEP-6 | 80 | CPI-210S | 1 | CELLOXIDE | 5 |

The details of the components shown in Tables 6 and 7 are as follows.

EPICLON 850: Bisphenol A type epoxy resin EPICLON 830: Bisphenol F type epoxy resin

EPICLON 3050: Bisphenol A type epoxy resin (higher molecular weight type than EPICLON 850)

EPICLON HP7200: Dicyclopentadiene type epoxy resin

EPICLON HP4700: Naphthalene type epoxy resin

EPICLON N-680: Cesol novolac type epoxy resin

EPICLON N-775: Phenol novolac type epoxy resin

All of these epoxy resins are manufactured by DIC Corporation.

BTHB-epo: Compound obtained in the above-mentioned Synthesis Example A1

CPI-2105: Photocationic initiator manufactured by San-Apro Ltd., cation structure: triarylsulfonium, anion: phosphorus-based

CELLOXIDE: CELLOXIDE 2021P (3′,4′-epoxycyclohexylmethyl-3,4-epoxycyclohexanecarboxylate, manufactured by Daicel Chemical Industries, Ltd.)

DY-BP: Butyl glycidyl ether

FAEP-6: 3-(perfluorohexyl)propene-1,2-oxide

These are reagents manufactured by Tokyo Chemical Industry Co., Ltd.

<Measurement and Evaluation>

(Viscosity)

Measurement was carried out at 25° C. and a shear rate of 100 (1/s) using a rheometer (Physica MCR51, manufactured by Anton Paar GmbH).

(Preparation of Cured Film for Evaluation)

The photocurable adhesive of each of Examples and Comparative Examples was applied to a glass substrate or a PET substrate (which one is used is described in each evaluation method below) using a bar coder so that the film thickness was 100 μm. As a result, a coating film was formed.

(As the glass substrate, a non-alkali glass substrate was used. As the PET substrate, a LUMIRROR (registered trademark) film having a thickness of 50 μm, manufactured by Toray Industries, Inc., was used.)

Then, the coating film was irradiated with light of 6000 mJ/cm² using an LED lamp emitting light having a wavelength of 365 nm.

In this manner, a cured film for evaluation was obtained.

(Photocurability)

Whether or not the cured film that was sufficiently cured after light irradiation was evaluated by tackiness. That is, the glass substrate with the cured film after light irradiation was sandwiched between tweezers, and whether or not the marks were left and whether or not the glass substrate was sticky were evaluated.

In the table shown below, the symbols in the column “Curability” mean the following:

◯ (Good): No tweezer marks on the cured film and no stickiness.

Δ (Insufficient): Tweezer marks are left to the cured film, and there is no stickiness.

X (Unacceptable): Tweezer marks are left on the cured film, and the glass substrate is sticky.

(Refractive Index)

The refractive index of the cured film formed on the glass substrate was measured at a wavelength of 632 nm using a prism coupler (2010/M, manufactured by Metricon Corporation). In addition, the refractive index of only the glass substrate was separately measured. Then, the refractive index of the cured film excluding the influence of the glass substrate was obtained.

(Adhesion)

The cured film formed on the substrate was subjected to a cross-cut test specified in JIS K 5600 5-6. Those with a classification of 0 specified by JIS were evaluated as ◯ (good adhesion), and those with classifications other than ◯ were evaluated as X (poor adhesion).

The evaluation was performed on both the glass substrate and the PET substrate.

(Bending Resistance)

The cured film prepared on the PET substrate was wound around a SUS tube having a diameter of 4 mm together with the PET film with the cured film on the outside, and the operation of bending by 180° was repeated 5 times. Then, the state of the cured film was visually observed and evaluated in the following four stages.

∞ (Very good): No cracks or wrinkles.

◯ (Good): There are some wrinkles.

Δ (Slightly poor): The film is not broken, but there are noticeable wrinkles.

X (Poor): The film does not maintain its state as a film, such as a partially broken film.

The measurement and evaluation results are summarized in the following tables “Evaluation of Adhesive Composition (Examples)” and “Evaluation of Adhesive Composition (Comparative Examples)”.

TABLE 8
Evaluation of adhesive composition (Examples)

| | Liquid | | | | | | | |
|---|---|---|---|---|---|---|---|---|
| | regulating | Viscosity | | Refractive | Transmittance | Adhesion | | Bending |
| Example | properties | (mPa · s) | Curability | index | (at 400 nm) | Glass | PET | resistance |
| 5-1 | ◯ | 600 | ◯ | 1.529 | 95 | ◯ | ◯ | ◯ |
| 5-2 | ◯ | 400 | ◯ | 1.521 | 95 | ◯ | ◯ | ◯◯ |
| 5-3 | ◯ | 280 | ◯ | 1.516 | 95 | ◯ | ◯ | ◯◯ |
| 5-4 | ◯ | 200 | ◯ | 1.511 | 95 | ◯ | ◯ | ◯ |
| 5-5 | ◯ | 150 | ◯ | 1.505 | 95 | ◯ | ◯ | ◯ |
| 5-6 | ◯ | 300 | ◯ | 1.513 | 95 | ◯ | ◯ | ◯◯ |
| 5-7 | ◯ | 350 | ◯ | 1.518 | 95 | ◯ | ◯ | ◯◯ |
| 5-8 | ◯ | 750 | ◯ | 1.516 | 95 | ◯ | ◯ | ◯ |
| 5-9 | ◯ | 890 | ◯ | 1.498 | 96 | ◯ | ◯ | ◯ |
| 5-10 | ◯ | 2200 | ◯ | 1.524 | 93 | ◯ | ◯ | ◯ |
| 5-11 | ◯ | 1900 | ◯ | 1.511 | 94 | ◯ | ◯ | ◯ |
| 5-12 | ◯ | 2000 | ◯ | 1.519 | 94 | ◯ | ◯ | ◯ |

TABLE 9
Evaluation of adhesive composition (Comparative Examples)

| | Liquid | | | | | | | |
|---|---|---|---|---|---|---|---|---|
| Comparative | regulating | Viscosity | | Refractive | | Adhesion | | Bending |
| Example | properties | (mPa · s) | Curability | index | Transmittance | Glass | PET | resistance |
| 2-1 | D | 520 | D | 1.572 | 94 | X | X | X |
| 2-2 | ◯ | 400 | D | 1.581 | 94 | X | X | X |
| 2-3 | X | — | — | — | — | — | — | — |
| 2-4 | D | 180 | X | — | — | — | — | — |

From Examples 5-1 to 5-12, it was shown that the photocurable adhesive including the compound (P), the first compound, and the photocationic initiator was useful as a photocurable adhesive and exhibited preferable performance, such as bending resistance, in the production of a foldable display.

### Additional Examples Relating to Polymers

1. Synthesis of BTHB-epo Derivative

(Synthesis of BTHB-epo-A)

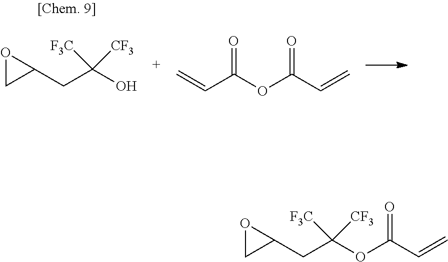

In a 300 mL three-necked eggplant flask, BTHB-epo (22.41 g, 100 mmol) and toluene (20 mL) were placed, mixed, and then ice-cooled. After being sufficiently ice-cooled, methanesulfonic acid (0.48 g, 5 mmol) was added to the flask, and then acrylic acid anhydride (12.61 g, 100 mmol) was added thereto. Then, the mixture was stirred at 80° C. for 8 hours.

After cooling the reaction solution, the solution was washed with a 10% by mass sodium hydroxide aqueous solution once, distillation washing was performed once, and then the solution was concentrated with an evaporator. In this manner, BTHB-epo-A (14.0 g, yield 50%) represented by the structural formula on the right side of the above chemical reaction formula was obtained.

¹H-NMR (CDCl₃): 6.50 ppm (br, 1H), 6.14 ppm (br, 1H), 5.90 ppm (br, 1H), 3.22 ppm (br, 1H), 2.97 ppm (t, 4.4 Hz, 1H), 2.53 ppm (dd, 4.4 Hz, 2.4 Hz, 1H), 2.46 ppm (dd, 15.2 Hz, 3.6 Hz, 1H), 1.81 ppm (ddd, 15.2 Hz, 8.8 Hz, 1.6 Hz, 1H)

¹⁹F-NMR: −73.2 ppm (q, 12.4 Hz, 6F)

2. Production of BTHB-epo-Containing Fluororesins 1 to 5

Before showing production examples of each resin (polymer), a method for measuring various characteristics of the resin (polymer) will be described.

[Measurement of Molar Ratio of Each Structural Unit]

The molar ratio of each structural unit in the polymer was determined from the measured value of ¹H-NMR, ¹⁹F-NMR or ¹³C-NMR.

[Measurement of Molecular Weight of Polymer]

The weight average molecular weight Mw and the molecular weight dispersity (a ratio of number average molecular weight Mn and weight average molecular weight Mw; Mw/Mn) of the polymer were measured using high-speed gel permeation chromatography (hereinafter, sometimes referred to as GPC, Model HLC-8320GPC, manufactured by Tosoh Corporation), in such a manner as to connect an ALPHA-M column and an ALPHA-2500 column (both manufactured by Tosoh Corporation) in series one by one and to use tetrahydrofuran (THF) as a developing solvent. As the detector, a refractive index difference measuring detector was used. In addition, polystyrene was used as a standard substance.

[Polymerization of BTHB-epo-Containing Fluororesin 1]

In a 300 mL three-necked eggplant flask, BTHB-epo (22.41 g, 100 mmol) (manufactured by Central Glass Co., Ltd.), and a fluorine-based solvent HFE 7300 (20 mL) manufactured by 3M Company were placed, mixed, and then ice-cooled. After being sufficiently ice-cooled, boron trifluoride diethyl ether complex (0.14 g, 1 mmol) was placed in the flask, the temperature was gradually raised, and the mixture was stirred overnight at 35° C.

When 100 g of n-heptane was added dropwise to the reaction system, a transparent viscous substance was precipitated. Then, the isolated viscous substance was isolated by decantation. Then, drying was performed under reduced pressure at 60° C.

From the above, 20 g of BTHB-epo-containing fluororesin 1 as a transparent viscous substance was obtained in a yield of 89%.

GPC measurement result: Mw=6,100, Mw/Mn=1.2

[Polymerization of BTHB-epo-Containing Fluororesin 2]

In a 300 mL three-necked eggplant flask, BTHB-epo (11.20 g, 50 mmol), BTHB-epo-A (13.96 g, 50 mmol) and HFE 7300 (24 mL) were placed, mixed, and then ice-cooled. After being sufficiently ice-cooled, a boron trifluoride diethyl ether complex (0.14 g, 1 mmol) was mixed in the flask, the temperature was gradually raised, and then the mixture was stirred overnight at 35° C.

When 100 g of n-heptane was added dropwise to the reaction system, a transparent viscous substance was precipitated. This viscous substance was isolated by decantation. Then, the isolated viscous substance was dried under reduced pressure at 60° C.

As a result, 22.6 g of BTHB-epo-containing fluororesin 2 was obtained as a transparent viscous substance in a yield of 90%.

GPC measurement result: Mw=6,900, Mw/Mn=1.3

NMR measurement result: The content ratio of the structural unit derived from BTHB-epo to the structural unit derived from BTHB-epo-A was 50.5:49.5 in terms of mol %.

[Polymerization of BTHB-epo-Containing Fluororesin 3]

In a 300 mL three-necked eggplant flask, BTHB-epo-containing fluororesin 1 (11.20 g, 50 mmol) and PGMEA (22 mL) were placed and mixed, and triethylamine (0.50 g, 5 mmol) was further placed in the flask and mixed. Then, 4-hydroxybutyl acrylate glycidyl ether (abbreviation: 4HBAGE, 5.0 g, 25 mmol) (manufactured by Mitsubishi Chemical Corporation) was added, the temperature was gradually raised, and the mixture was stirred overnight at 85° C.

After cooling, 110 g of n-heptane was added dropwise to the reaction system, and a transparent viscous substance was precipitated. This viscous substance was isolated by decantation. Then, the isolated viscous substance was dried under reduced pressure at 60° C.

As a result, 11.34 g of BTHB-epo-containing fluororesin 3 was obtained as a transparent viscous substance in a yield of 70%.

GPC measurement result: Mw=7,900, Mw/Mn=1.4

NMR measurement result: The content ratio of the structural unit derived from BTHB-epo to the structural unit derived from BTHB-epo into which 4HBAGE was introduced was 52.5:47.5 in terms of mol %.

[Polymerization of BTHB-epo-Containing Fluororesin 4]

In a 300 mL three-necked eggplant flask, BTHB-epo-containing fluororesin 1 (11.20 g, 50 mmol) and PGMEA (22 mL) were placed, mixed, and ice-cooled, and then triethylamine (0.50 g, 5 mmol) was placed in the flask and mixed. Then, Calends AOI (3.5 g, 25 mmol) (manufactured by Showa Denko K.K.) was gradually added, and then the temperature was raised, and the mixture was stirred overnight at 55° C.

When 90 g of n-heptane was added dropwise to the reaction system, a transparent viscous substance was precipitated. This viscous substance was isolated by decantation. The isolated viscous substance was dried under reduced pressure at 60° C.

As described above, 9.6 g of BTHB-epo-containing fluororesin 4 as a transparent viscous substance was obtained in a yield of 65%.

GPC measurement result: Mw=7,600, Mw/Mn=1.3

NMR measurement result: The content ratio of the structural unit derived from BTHB-epo to the structural unit derived from BTHB-epo into which Calends AOI was introduced was 52.0:48.0 in terms of mol %.

[Polymerization of BTHB-epo-Containing Fluororesin 5]

In a 300 mL three-necked eggplant flask, BTHB-epo-containing fluororesin 1 (11.20 g, 50 mmol) and PGMEA (22 mL) were placed and mixed, and then triethylamine (0.50 g, 5 mmol) was mixed. Then, 4HBAGE (3.0 g, 15 mmol, manufactured by Mitsubishi Chemical Corporation) and 3-perfluorobutyl-1,2-epoxypropane (hereinafter referred to as C4F9-epo, 4.2 g, 15 mmol, reagent manufactured by Tokyo Chemical Industry Co., Ltd.) were added into the flask, the temperature was gradually raised, and the mixture was stirred overnight at 85° C.

After cooling, 150 g of n-heptane was added dropwise to the reaction system, and a transparent viscous substance was precipitated. This viscous substance was isolated by decantation. The isolated viscous substance was dried under reduced pressure at 60° C.

As described above, 13.8 g of the BTHB-epo-containing fluororesin 5 as a transparent viscous substance was obtained in a yield of 75%.

GPC measurement result: Mw=8,900, Mw/Mn=1.5

NMR measurement result: The content ratio of the structural unit derived from BTHB-epo, the structural unit derived from BTHB-epo to which 4HBAGE was introduced, and the structural unit derived from BTHB-epo into which C4F9-epo was introduced was 40.5:30.0:29.5 in terms of mol %.

3. Preparation of Photosensitive Resin Composition

[Preparation of Photosensitive Resin Composition 1]

30 parts by mass of the produced BTHB-epo-containing fluororesin 2, 0.3 parts by mass of IRGACURE OXE01 (a product manufactured by BASF) as a photopolymerization initiator, and 70 parts by mass of propylene glycol monomethyl ether acetate (PGMEA) as a solvent were blended to obtain a solution. Then, the obtained solution was filtered through a membrane filter having a pore size of 0.2 μm. In this manner, photosensitive resin composition 1 was prepared.

[Preparation of Photosensitive Resin Composition 2]

30 parts by mass of the produced BTHB-epo-containing fluororesin 3, 0.3 parts by mass of IRGACURE OXE01 (a product manufactured by BASF) as a photopolymerization initiator, and 70 parts by mass of propylene glycol monomethyl ether acetate (PGMEA) as a solvent were blended to obtain a solution. Then, the obtained solution was filtered through a membrane filter having a pore size of 0.2 μm. In this manner, photosensitive resin composition 2 was prepared.

[Preparation of Photosensitive Resin Composition 3]

30 parts by mass of the produced BTHB-epo-containing fluororesin 4, 0.3 parts by mass of IRGACURE OXE01 (a product manufactured by BASF) as a photopolymerization initiator, and 70 parts by mass of propylene glycol monomethyl ether acetate (PGMEA) as a solvent were blended to obtain a solution. Then, the obtained solution was filtered through a membrane filter having a pore size of 0.2 μm. In this manner, photosensitive resin composition 3 was prepared.

[Preparation of Photosensitive Resin Composition 4]

30 parts by mass of the produced BTHB-epo-containing fluororesin 5, 0.3 parts by mass of IRGACURE OXE01 (a product manufactured by BASF) as a photopolymerization initiator, and 70 parts by mass of propylene glycol monomethyl ether acetate (PGMEA) as a solvent were blended to obtain a solution. Then, the obtained solution was filtered through a membrane filter having a pore size of 0.2 μm. In this manner, photosensitive resin composition 4 was prepared.

4. Evaluation

Using the photosensitive resin compositions 1 to 4 obtained in the above “3. Preparation of Photosensitive Resin Composition”, the patterning performance was evaluated as follows. The results are shown in Table 1.

[Pattern Formation]

First, a circular non-alkali substrate having a diameter of 10 cm was washed with ultrapure water and then acetone, and then an UV ozone treatment was performed on the substrate for 5 minutes using a UV ozone treatment device.

Next, each of the photosensitive resin compositions 1 to 4 obtained in “3. Preparation of Photosensitive Resin Composition” was applied to the substrate after the UV ozone treatment at a rotation speed of 1,000 rpm using a spin coater. Then, heating was performed at 70° C. for 120 seconds on a hot plate to form a fluororesin film having a film thickness of 5 μm.

Using a mask aligner (a product manufactured by Suss Microtec k.k.), the obtained resin film was irradiated with an i-ray (wavelength: 365 nm) through the mask having a line-and-space of 10 μm, and exposed.

The resin film after exposure was evaluated for developer solubility and patterning (sensitivity, resolution) as follows.

[Developer Solubility]

The exposed resin film on the glass substrate was immersed in an alkaline developer at room temperature for 60 seconds to evaluate the solubility in the alkaline developer. A 2.38% by mass tetramethylammonium hydroxide aqueous solution (hereinafter, may be referred to as TMAH) was used as the alkaline developer. The solubility was evaluated by measuring the film thickness of the coating film after immersion with a contact type film thickness meter. A case where the resin film after exposure was completely dissolved was defined as “soluble”, and a case where at least a part of the resin film after exposure remained undissolved was defined as “insoluble”.

[Patterning Performance (Sensitivity, Resolution)]

First, in the above [Pattern Formation], the optimum exposure amount Eop (mJ/cm²) for forming a line-and-space pattern was obtained and used as an index of sensitivity. The “optimal exposure amount Eop” refers to an exposure amount that can form a pattern of line/space=10 μm/10 μm as it is when a mask of line/space=10 μm/10 μm is used.

In addition, the obtained pattern was observed with a microscope and the resolution was evaluated. Those for which line edge roughness cannot be confirmed were rated as “excellent”, those for which line edge roughness can be slightly confirmed were rated as “good”, and those for which line edge roughness are remarkable were rated as “unacceptable”.

[Evaluation of Refractive Index of Cured Film]

A cured film without a pattern was prepared by performing the same operation except that the film was exposed without using the mask in the above [Pattern formation]. The refractive index of the obtained cured film was measured using a prism coupler (2010/M, manufactured by Metricon Corporation, wavelength: 632 nm).

[Evaluation of Adhesion of Cured Film (Pattern)]

A patterned substrate was prepared by performing the same operation except that a silicon wafer and a polyethylene terephthalate substrate were used as the substrate described in [Pattern Formation] above, in addition to the glass substrate.

A cross-cut test (JIS K 5600 5-6) was performed on the cured film (pattern) formed on each substrate. As for the determination, a film whose cross-cut test result was classification of 0 was rated as good (◯), and the others were rated as poor (X).

The results of each of the above evaluations are summarized in the table below.

### [Table 10]

TABLE 10

| Photosensitive resin | | 1 | 2 | 3 | 4 |
|---|---|---|---|---|---|
| composition | | | | | |
| BTHB-epo-containing | | 2 | 3 | 4 | 5 |
| fluororesin | | | | | |
| Developer | Unexposed | Soluble | Soluble | Soluble | Soluble |
| | portion | | | | |
| solubility | Exposed | Insoluble | Insoluble | Insoluble | Insoluble |
| | portion | | | | |
| Pattern | Sensitivity | 110 | 105 | 100 | 150 |
| | (mJ/ cm2) | | | | |
| performance | Resolution | Excellent | Excellent | Excellent | Excellent |
| Refractive | Wavelength | 1.39 | 1.41 | 1.40 | 1.37 |
| index | 632 nm | | | | |
| Adhesion | To glass | ◯ | ◯ | ◯ | ◯ |
| | To Si wafer | ◯ | ◯ | ◯ | ◯ |
| | To PET | ◯ | ◯ | ◯ | X |

As shown in the above table, the photosensitive resin compositions 1 to 4 exhibited good developer solubility and patternability. In addition, the refractive index and adhesion of each film formed by using the photosensitive resin compositions 1 to 4 were good.

Incidentally, it is considered that the reason why the adhesion of the photosensitive resin composition 4 to PET is X is that the BTHB-epo-containing fluororesin 5 has a structural unit derived from C4F9-epo (there is a possibility that the interaction with PET may be reduced due to the effect of perfluorobutyl group).

This application claims priority based on Japanese Patent Application No. 2020-019398 filed on Feb. 7, 2020, and Japanese Application Japanese Patent Application No. 2020-150682 filed on Sep. 8, 2020, the entire contents of which are incorporated herein by reference.

1. A curable composition comprising:
a first compound having a group represented by general formula (x) and an epoxy group, and having a molecular weight of 1000 or less,
—C(Rf₁)(Rf₂)—OH (x),
in the general formula (x), Rf₁ and Rf₂ each independently represent a fluorine-containing alkyl group.

2. The curable composition according to claim 1,
wherein the first compound includes a compound represented by general formula (1), 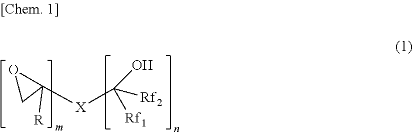
in the general formula (1),
R represents a hydrogen atom or a monovalent organic group,
X represents a (m+n)-valent organic group,
m represents 1 to 4,
n represents 1 to 4, and
Rf₁ and Rf₂ each independently represent a fluorine-containing alkyl group.

3. The curable composition according to claim 1,
wherein a fluorine atom content in the first compound is 25% to 60% by mass.

4. The curable composition according to claim 1,
wherein Rf₁ and Rf₂ each represent a fluoroalkyl group having 1 to 10 carbon atoms.

5. The curable composition according to claim 4,
wherein the fluoroalkyl group is a perfluoroalkyl group.

6. The curable composition according to claim 5,
wherein the perfluoroalkyl group is a trifluoromethyl group.

7. The curable composition according to claim 2,
wherein X in the general formula (1) represents an alkylene group.

8. The curable composition according to claim 2,
wherein X in the general formula (1) represents a methylene group.

9. The curable composition according to claim 1, further comprising:
a cationic polymerization initiator.

10. The curable composition according to claim 9,
wherein the cationic polymerization initiator includes a photocationic polymerization initiator.

11. The curable composition according to claim 1, further comprising:
a second compound that is different from the first compound and has a group capable of forming a covalent bond by reacting with the epoxy group in the first compound.

12. The curable composition according to claim 11,
wherein the second compound includes a monofunctional epoxy compound.

13. The curable composition according to claim 11,
wherein the second compound includes a polyfunctional epoxy compound.

14. The curable composition according to claim 10, further comprising:
a compound (P) which has an aromatic ring skeleton and/or an alicyclic skeleton and in which a total of the number of hydroxy groups and the number of epoxy groups per molecule is 2 or more.

15. The curable composition according to claim 14,
wherein the compound (P) includes one or more epoxy resins selected from the group consisting of a bisphenol A type epoxy resin, a bisphenol F type epoxy resin, a dicyclopentadiene type epoxy resin, a naphthalene type epoxy resin, a cresol novolac type epoxy resin, and a phenol novolac type epoxy resin.

16. The curable composition according to claim 14,
wherein the number of moles of the first compound included in 100 g of a non-volatile component is 0.04 to 0.4 mol.

17. The curable composition according to claim 14,
wherein a viscosity is 100 to 6000 mPa·s.

18. The curable composition according to claim 14,
wherein a light transmittance of a cured film having a film thickness of 100 μm, obtained by curing the curable composition, at a wavelength of 400 nm is 90% or more.

19. A cured product of the curable composition according to claim 1.

20. An electronic device comprising:
the cured product according to claim 19.

21. A display device comprising:
the cured product according to claim 19.

22. An optical member comprising:
the cured product according to claim 19.

23. A polymer comprising:
a structural unit represented by general formula (2), 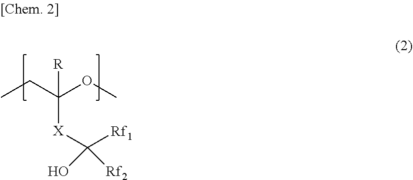
in the general formula (2),
R represents a hydrogen atom or a monovalent organic group,
X represents a divalent organic group,
Rf₁ represents a fluorine-containing alkyl group, and
Rf₂ represents a fluorine-containing alkyl group.

24. The polymer according to claim 23, further comprising:
a structural unit represented by general formula (3), 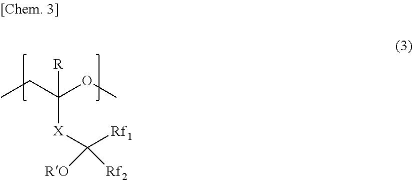
in the general formula (3),
R′ represents a monovalent organic group, and
definitions of X, Rf₁, and Rf₂ are the same as those in the general formula (2).

25. The polymer according to claim 24,
wherein R′ includes a polymerizable group.

26. A photosensitive composition comprising:
the polymer according to claim 24;
a photopolymerization initiator; and
a solvent.

27. A pattern formed using the photosensitive composition according to claim 26.

28. A compound represented by general formula (1), 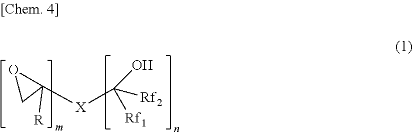
in the general formula (1),
R represents a hydrogen atom or a monovalent organic group,
X represents a (m+n)-valent organic group,
m represents 1 to 4,
n represents 1 to 4, and
Rf₁ and Rf₂ each independently represent a fluorine-containing alkyl group.
